(19) United States
(12) Patent Application Publication
(10) Pub. No.: US 2008/0182880 A1
(43) Pub. Date: Jul. 31, 2008

(54) PIOGLITAZONE COMPOSITION

(76) Inventors: Mailatur Sivaraman Mohan, Hyderabad (IN); Indu Bhushan, Hyderabad (IN); Sivareddy Venkata Pallempalli, Hyderabad (IN); Kamlakar Golli Reddy, Warangal (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/862,577

(22) Filed: Sep. 27, 2007

Related U.S. Application Data
(60) Provisional application No. 60/884,935, filed on Jan. 15, 2007.

(30) Foreign Application Priority Data
Sep. 28, 2006 (IN) 1805/CHE/2006

(51) Int. Cl. A61K 31/4439; C07D 417/02

(52) U.S. Cl. 514/342; 546/269.7

(57) ABSTRACT

Pioglitazone or a salt thereof having a particle size distribution wherein D₉₀ is about 25 μm to about 40 μm, and pharmaceutical compositions prepared therefrom.

The present invention relates to a physical form of pioglitazone or its pharmaceutically acceptable salts and oral solid dosage forms containing pioglitazone or a pharmaceutically acceptable salt thereof of defined physical form.

Pioglitazone has a chemical name (±)-5-[[4-[2-(5-ethyl-2-pyridinyl)ethoxy]phenyl]methyl]-2,4-]thiazolidinedione. It belongs to a different chemical class and has as a different pharmacological action than that of the sulfonylureas, metformin, or the α-glucosidase inhibitors. The molecule contains one asymmetric carbon and is used as the racemic mixture. The two enantiomers of pioglitazone interconvert in vivo. No differences have been found in pharmacological activity between the two enantiomers. Pioglitazone hydrochloride is an odorless white crystalline powder that has a molecular formula of C₁₉H₂₀N₂O₃S.HCl and a molecular weight of 392.90 daltons. It is soluble in N,N-dimethylformamide, slightly soluble in anhydrous ethanol, very slightly soluble in acetone and acetonitrile, practically insoluble in water, and insoluble in ether.

The structural formula of pioglitazone hydrochloride (1) is:

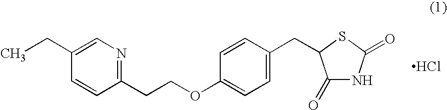

The pharmaceutically acceptable salts of pioglitazone are exemplified by salts with inorganic bases, salts with organic bases, salts with inorganic acids, salts with organic acids and salts with basic or acidic amino acids. Examples of salts with inorganic bases included but not limited to salts with alkali metals such as calcium, magnesium, etc., and salts with aluminium, ammonium, etc. Examples of salts with organic bases include but not limited to salts with trimethylamine, triethylamine, pyridine, picoline, ethanolamine, diethanolamine, triethanolamine, dicyclohexylamine, N,N-dibenzylethylenediamine, etc. Examples of salts with inorganic acids include but are not limited to salts with hydrochloric acid, hydrobromic acid, nitric acid, sulfuric acid, phosphoric acid, etc. Examples of salts with organic acids include but are not limited to salts with formic acid, acetic acid, trifluoroacetic acid, fumaric acid, oxalic acid, tartaric acid, maleic acid, citric acid, succinic acid, methanesulfonic acid, benzenesulfonic acid, p-toluenesulfonic acid, etc. Examples of salts with basic amino acids include but are not limited to salts with arginine, lysine, ornithine, etc. and examples of salts with acidic amino acids include but are not limited to salts with aspartic acid, glutamic acid, etc. The amount of pioglitazone in a solid pharmaceutical composition is not particularly limited and comprises any amount that is pharmaceutically effective.

Pioglitazone hydrochloride is currently marketed as ACTOS® tablets for oral administration and is available in strengths of 15 mg, 30 mg and 45 mg of pioglitazone (expressed as the base) formulated with the following excipients: lactose monohydrate NF, hydroxypropylcellulose NF, carboxymethylcellulose calcium NF, and magnesium stearate NF.

Pioglitazone is an oral antihyperglycemic agent that acts primarily by decreasing insulin resistance. Pharmacological studies indicate that pioglitazone improves sensitivity to insulin in muscle and adipose tissue and inhibits hepatic gluconeogenesis. Pioglitazone is a potent and highly selective agonist for peroxisome proliferator-activated receptor-gamma (PPARγ). PPAR receptors are found in tissues important for insulin action such as adipose tissue, skeletal muscle, and liver. Activation of PPARγ nuclear receptors modulates the transcription of a number of insulin responsive genes involved in the control of glucose and lipid metabolism. Pioglitazone improves glucose resistance while reducing circulating insulin levels and is useful in the treatment of diabetes, particularly type II diabetes, also known as non-insulin dependent diabetes mellitus (NIDDM) or adult onset diabetes. Type II diabetes is known as a disease characterized by insulin resistance.

Pioglitazone is disclosed in U.S. Pat. No. 4,687,777. U.S. Pat. No. 5,952,509, incorporated herein by reference, discloses methods for the synthesis of pioglitazone. U.S. Pat. Nos. 5,965,584 and 6,329,404 disclose pharmaceutical compositions comprising insulin sensitivity enhancers in combination with one or more other antidiabetic agents. U.S. Pat. Nos. 6,150,384, 6,166,042, 6,172,090, 6,166,043, 6,211,205, 6,271,243, and 6,303,640 disclose methods of treatment using pioglitazone.

International Application Publication No. WO 2003/080056, U.S. Patent Application Publication No. 2005/0131027, and European Patent Application No. 1465628 disclose dosage forms of pioglitazone with defined particle size distributions.

U.S. Pat. No. 6,740,339 and International Application Publication No. WO 2000/078292 disclose quickly disintegrating solid preparations. International Application Publication Nos. WO 2005/099760, WO 2004/078175, WO 2004/069229, and WO 2004/006921 disclose various pharmaceutical compositions of pioglitazone.

Particle size can affect the solubility properties of pioglitazone. However it is not known how to define the limits of particle size properties required in order to provide appropriate bioavailability from a solid dosage form containing pioglitazone. In view of the foregoing, there is need in the medical arts for pioglitazone with a defined particle size and improved bioavailability.

### SUMMARY OF THE INVENTION

The present invention relates to a physical form of pioglitazone or its pharmaceutically acceptable salts, and oral solid dosage forms containing pioglitazone or a pharmaceutically acceptable salt thereof of defined physical form.

In one of the embodiments the invention includes defined physical forms of pioglitazone or a pharmaceutically acceptable salt thereof having a defined particle size distribution.

In another embodiment the invention includes pharmaceutical compositions comprising pioglitazone or a pharmaceutically acceptable salt thereof of defined physical form and at least one pharmaceutically acceptable excipient.

In another embodiment the invention includes processes for preparing a pharmaceutical composition.

In another embodiment the invention includes methods of using the pharmaceutical composition.

An embodiment of the invention provides pioglitazone or a salt thereof having a particle size distribution wherein D₉₀ is about 25 μm to about 40 μm.

Another embodiment of the invention provides pioglitazone or a salt thereof having a particle size distribution wherein D₉₀ is about 25 μm to about 40 μm, D₅₀ is about 8 μm to about 16 μm, D₁₀ is about 1 μm to about 4 μm, and D_{[4,3]} is about 12 μm to about 17 μm.

A further embodiment of the invention provides a pharmaceutical formulation comprising pioglitazone hydrochloride having a particle size distribution wherein D₉₀ is about 25 μm to about 40 μm, D₅₀ is about 8 μm to about 16 μm, D₁₀ is about 1 μm to about 4 μm, and D_{[4,3]} is about 12 μm to about 17 μm, and at least one pharmaceutical excipient, wherein at least about 70% of contained pioglitazone dissolves within about 30 minutes upon immersion in a buffer having a pH about 2.

### DETAILED DESCRIPTION

The present invention relates to physical forms of pioglitazone or a pharmaceutically acceptable salt thereof, and oral solid dosage forms containing pioglitazone or a pharmaceutically acceptable salt thereof of defined physical form.

In one of the embodiments the invention includes physical forms of pioglitazone or a salt thereof having a defined particle size distribution.

In another embodiment the invention includes processes for preparing pharmaceutical compositions.

In another embodiment the invention includes methods of using the pharmaceutical compositions.

Particle size reduction increases the surface area of the solid phase that is in contact with a liquid medium. This particle size distributions according to the present invention provide an enhanced rate of dissolution of the pioglitazone and provide reproducible bioavailability. The pioglitazone of the invention can also be incorporated into oral dosage forms such as tablets or capsules, etc. to enhance the physicochemical properties desired. The preferred rates of dissolution and absorption herein provide for early onset of pioglitazone absorption, yet avoid very high and rapidly achieved plasma drug concentrations. A very high and rapidly achieved concentration can lead to undesirable hypoglycemia. The pioglitazone of the physical forms described herein achieves rapid onset of action, yet also maintains exposure of the patient to drug (as measured by the rate under the plasma drug concentration versus time curve), and therefore maintains the efficacy of the formulation.

There are instances where the rate of dissolution of a poorly soluble drug is a rate-limiting factor in its absorption by the body. It is recognized that such drugs may be more readily bioavailable if administered in a finely divided state. Because of the poor water solubility of pioglitazone the rate of dissolution of drug from a dosage form is a controlling factor in determining the rate and extent of drug absorption. The rate of dissolution depends on factors including particle size (or particle surface area, which can be related to particle size). It has been found that an appropriate bioavailability for pioglitazone is obtained when the particle size reduction of the pioglitazone is controlled so as not to provide what is classically accepted as “micronized” material, yet is fine enough to provide for desired rates of dissolution.

Particle size also can affect how freely crystals or a powdered form of a drug will flow, which has consequences in the production processing of pharmaceutical products containing the drug.

The percent of particles with different dimensions that exist in a powder is called the particle size distribution. It is represented in certain ways. Particle size is the maximum dimension of a particle, normally expressed in units of μm. Particle size distributions can be expressed in terms of, D₁₀, D₅₀, D₉₀ and D_{[4,3]}. The D₁₀, D₅₀ and D₉₀ represent the 10th, median or the 50th percentile, and the 90th percentile of the particle size distribution, respectively, as measured by volume. That is, the D₁₀, D₅₀, D₉₀ is a value of the distribution such that 10%, 50%, 90% by volume of the particles have a size of this value or less, or is the percentage of particles smaller than that size. D₅₀ is also known as median diameter of particle. It is one of the important parameters representing characteristics of particle of powder. For a sample, if D₅₀=5 μm, it means that 50% of the particles are smaller than 5 μm. Similarly, if D₁₀=5 μm, 10% by volume of the particles are less than or equal to 5 μm, and if D₉₀=5 μm, 90% of the particles are less than or equal to 5 μm. D_{[4,3]} means the volume moment mean of the particle or the volume weighted particle size.

In one of the embodiments, the invention includes a defined physical form of pioglitazone or a salt thereof, having a defined particle size distribution. The defined particle size includes a plurality of pioglitazone particles wherein the D₅₀ (mean particle size) is about 8 μm to about 16 μm, D₁₀ is about 1 μm to about 4 μm, Dgo is about 25 μm to about 40 μm, and D_{[4,3]} is about 12 μm to about 17 μm.

In another embodiment the present invention includes to pioglitazone or a salt thereof of defined particle size including a plurality of pioglitazone particles obtained by communication using a fluid energy mill, wherein the D₅₀ (mean particle size) is about 8 μm to about 16 μm, D₁₀ is about 1 μm to about 4 μm, D₉₀ is about 25 μm to about 40 μm, and D_{[4,3]} is about 12 μm to about 17 μm.

In an embodiment the present invention includes a pharmaceutical composition including pioglitazone or a salt thereof of defined particle size wherein the D₅₀ (mean particle size) is about 8 μm to 16 μm, D₁₀ is about 1 μm to about 4 μm, D₉₀ is about 25 μm to about 40 μm, and D_{[4,3]} is about 12 μm to 17 μm, together with at least one pharmaceutically acceptable excipient.

In another embodiment the invention includes pharmaceutical formulations comprising pioglitazone or a salt thereof of defined particle size, wherein at least about 70% of contained pioglitazone dissolves within about 30 minutes upon immersion in a buffer having pH about 2.

In another embodiment the invention includes the pharmaceutical formulations comprising pioglitazone or a salt thereof of defined particle size producing plasma Cₘₐₓ values ranging from about 1,000 ng/mL to about 1,700 ng/mL after administration of a single 45 mg pioglitazone dose to healthy humans.

In another embodiment the invention includes pharmaceutical formulations comprising pioglitazone or a salt thereof of defined particle size producing plasma AUC_{0-T} values about 10,000 ng·hour/mL to about 19,000 ng·hour/mL after administration of a single 45 mg pioglitazone dose to healthy humans.

In another embodiment the invention includes pharmaceutical formulations comprising pioglitazone or a salt thereof of defined particle size producing plasma AUC₀₋ₜ values about 12,000 ng·hour/mL to about 20,000 ng·hour/mL after administration of a single 45 mg pioglitazone dose to healthy humans.

Also helpful for obtaining the appropriate bioavailability is the choice of excipients used in the formulation. Useful excipients will be those that allow drug release to occur without substantially influencing the rate of drug dissolution and hence absorption. Such excipients will be highly soluble in water, and hence dissolve rapidly when the dosage forms immersed in an aqueous environment. In this way poorly soluble pioglitazone is liberated as a finely divided suspension. Dissolution of pioglitazone from this suspension, the rate of which is controlled by the particle size distribution of the suspension, is a prerequisite for absorption. Hence the absorption characteristics are defined by the particle size distribution of the pioglitazone. Poorly soluble excipients may result in dosage form that erodes too slowly.

The pharmaceutical composition of the present invention includes pioglitazone or a pharmaceutically acceptable salt thereof and at least one pharmaceutically acceptable excipient. Pharmaceutically acceptable excipients include but are not limited to diluents, disintegrants, binders, lubricants, colorants, stabilizers, pH modifiers, surfactants, artificial sweeteners, flavoring agents, and the like.

### Diluents

Various useful diluents include but are not limited to starches, lactose, mannitol, cellulose derivatives and the like. Different grades of lactose include but are not limited to lactose monohydrate, lactose DT (direct tableting), lactose anhydrous, Flowlac™ (available from Meggle Products), Pharmatose™ (available from DMV) and others. Different grades of starches included but are not limited to maize starch, potato starch, rice starch, wheat starch, pregelatinized starch (commercially available as PCS PC10 from Signet Chemical Corporation) and Starch 1500, Starch 1500 LM grade (low moisture content grade) from Colorcon, fully pregelatinized starch (commercially available as National 78-1551 from Essex Grain Products) and others. Different cellulose compounds that can be used include crystalline cellulose and powdered cellulose. Examples of crystalline cellulose products include but are not limited to CEOLUS™ KG801, Avicel™ PH 101, PH102, PH301, PH302 and PH-F20, microcrystalline cellulose 114, and microcrystalline cellulose 112. Other useful diluents include but are not limited to carmellose, sugar alcohols such as mannitol, sorbitol and xylitol, calcium carbonate, magnesium carbonate, dibasic calcium phosphate, and tribasic calcium phosphate.

### Disintegrants

Various useful disintegrants include but are not limited to carmellose calcium (Gotoku Yakuhin Co., Ltd.), carboxymethylstarch sodium (Matsutani Kagaku Co., Ltd., Kimura Sangyo Co., Ltd., etc.), croscarmellose sodium (FMC-Asahi Chemical Industry Co., Ltd.), crospovidone, examples of commercially available crospovidone products including but not limited to crosslinked povidone, Kollidon™ CL [manufactured by BASF (Germany)], Polyplasdone™ XL, XI-10, and INF-10 [manufactured by ISP Inc. (USA)], and low-substituted hydroxypropylcellulose. Examples of low-substituted hydroxypropylcellulose include but are not limited to low-substituted hydroxypropylcellulose LH11, LH21, LH31, LH22, LH32, LH20, LH30, LH32 and LH33 (all manufactured by Shin-Etsu Chemical Co., Ltd.). Other useful disintegrants include sodium starch glycolate, colloidal silicon dioxide, and starch.

### Binders

Various useful binders include but are not limited to hydroxypropylcellulose (Klucel™-LF), hydroxypropyl methylcellulose (Methocel™), polyvinylpyrrolidone (PVP-K25, PVP-K29, PVP-K30), powdered acacia, gelatin, guar gum, carbomer (e.g. carbopol), methylcellulose, polymethacrylates, and starch.

### Lubricants

Various lubricants that can be used include but are not limited to magnesium stearate, sucrose esters of fatty acids, polyethylene glycol, talc, stearic acid, and sodium stearyl fumarate.

### Colorants

Various useful colorants include but are not limited to Food Yellow No. 5, Food Red No. 2, Food Blue No. 2, and the like, food lake colorants, and ferric oxide.

### Stabilizers

Various useful stabilizers include but are not limited to disodium edetate, tocopherol, and cyclodextrins.

### pH Modifiers

Various pH modifiers that can be used include but are not limited to citrates, phosphates, carbonates, tartrates, fumarates, acetates, and amino acid salts.

### Surfactants

Various useful surfactants include but are not limited to sodium lauryl sulfate, polysorbate 80, hydrogenated oil, polyoxyethylene glycol, and polyoxypropylene glycol.

### Sweeteners

Various useful sweeteners include but are not limited to saccharin sodium, dipotassium glycylrrhizinate, aspartame, stevia, thaumatin, sucrose, fructose, mannitol, and invert sugar.

### Flavoring Agents

Various useful flavoring agents include but are not limited to lemon oil, orange oil, and menthol.

An embodiment of the invention includes pharmaceutical preparations made in accordance with the invention that are solid dosage forms, which include but are not limited to capsules, tablets, caplets, pills, powders, granules, etc. Solid compositions of a similar type may also be filled into soft and hard-filled gelatin capsules using such excipients as lactose or milk sugar, as well as high molecular weight polyethylene glycols and the like.

Solid dosage forms such as tablets, capsules, pills, and granules can be prepared with coatings and shells, such as enteric coatings and others well known in the art. They may contain opacifying agents, and can also be of such composition that they release the active compound or compounds in a certain part of the intestinal tract in a delayed manner. Examples of embedding compositions, which can be used, are polymeric substances and waxes. The active compounds can also be in microencapsulated form, if appropriate, with one or more of the above-mentioned excipients.

In an embodiment of the invention, pioglitazone or a salt thereof of the defined physical forms described herein can also be used in formulations further containing other drugs used in the treatment of Type II diabetes. Examples include but are not limited to acarbose or other glycosidase inhibitors, glyburide, rosiglitazone or other thaizolidones, biguanides such as metformin, repaglinide and other “aglinides”, sulfonylureas such as tolbutamide, chlorpropramide, tolazamide, acetohexamide, 4-chloro-N-[(1-pyrrolidinylamino)carbonyl]-benzenesulfonamide or its ammonium salt, glibenclamide, gliclazide, 1-butyl-3-metanilylurea, carbutamide, glibonuride, glizipide, gliquidone, glisoxepid, glybuthiazole, glibuzole, glyhexamide, glymidine, glypinamide, phenbutamide and tolcyclamide.

In another embodiment the solid oral dosage forms of the present invention will be formulated to provide a unit dose of pioglitazone about 5 to about 50 milligrams per individual dosage form.

The desired particle size distributions may be obtained by techniques such as sieving or air jet milling and can be measured by a laser light scattering method.

In another embodiment the invention includes the method of sorting by particle size involving passing the milled material through a stack of sieves, each with openings of a different size. The sieves are arranged so that the material encounters the sieve having the largest openings first and those particles that pass through the first sieve encounter a second sieve with smaller openings and those that pass through the second sieve may encounter a third sieve, etc. Pioglitazone particles can also be separated by particle size using cyclonic or centrifugation techniques.

In an embodiment the invention includes size distributions of pioglitazone particles as determined by laser diffraction. The sizes of pioglitazone particles reported herein were determined using a Malvern™ Mastersizer™ laser diffraction instrument (Malvern Instruments Ltd., Malvern, Worcestershire, UK). Samples of the pioglitazone were suspended in sunflower oil. The suspensions were mixed and then sonicated for 120 seconds to thoroughly disperse the pioglitazone particles. The dispersion was then circulated in the flow cell of the Malvern Mastersizer for two minutes before particle size measurements were taken.

In an embodiment the invention includes the use of packaging materials such as containers and lids of high-density polyethylene (HDPE), low-density polyethylene (LDPE) and or polypropylene and/or glass, and blisters or strips composed of aluminium or high-density polypropylene.

### Processes for Preparing Compositions

The present invention is further directed to processes for preparing pharmaceutical compositions comprising pioglitazone or a pharmaceutically acceptable salt thereof of defined particle size wherein the D₅₀ (mean particle size) is about 8 μm to 16 μm, D₁₀ is about 1 μm to about 4 μm, D₉₀ is about 25 μm to about 40 μm, and D_{[4,3]} is about 12 μm to 17 μm, together with at least one pharmaceutically acceptable excipient.

In one of the embodiments a method of preparing a pharmaceutical composition includes, but is not limited to, one or more of physical mixing, blending, dry granulation, wet granulation, and direct compression.

In an embodiment the present invention is directed to processes for preparing pharmaceutical compositions comprising an effective amount of pioglitazone or its pharmaceutically acceptable salt of defined particle size and at least one pharmaceutically acceptable excipient, comprising:

1) Sifting pioglitazone or its pharmaceutically acceptable salt of defined particle size, diluent, disintegrant, and binder through an appropriate mesh sieve.

2) Loading the sifted materials into a granulator and mixing for an appropriate time at a fast speed.

3) Preparing a granulating fluid.

4) Adding granulating fluid of step 3 to the blend of step 2 with the impeller at fast speed and chopper off, and unloading the granules into the bowl of a fluid bed drier.

5) Drying the wet granules of step 4.

6) Sifting the dried granules of step 5 through an appropriate mesh sieve.

7) Milling the sieve-retained particles of step 6 through an appropriate size screen.

8) Sifting the milled granules of step 7 through an appropriate mesh sieve.

9) Loading the sifted materials of step 6 and step 8, and magnesium stearate (sifted through an appropriate mesh sieve), into a blender and blending for an appropriate time.

10) Compressing the blend into solid dosage forms or filling into empty capsule shells.

The dosage forms can be subjected to an in vitro dissolution evaluation according to Test 711 “Dissolution” in United States Pharmacopoeia 24, United States Pharmacopeial Convention, Inc., Rockville, Md., 1999, (“USP”) to determine the rate at which the pioglitazone is released from the dosage forms, and pioglitazone concentrations can be determined in solutions by techniques such as high performance liquid chromatography. The pharmaceutical dosage forms of the present invention are intended for oral administration to a patient in need thereof.

In determining bioequivalence, for example, between two products such as a commercially available product and a proposed product, pharmacokinetic studies are conducted whereby each of the preparations is administered in a crossover study to volunteer subjects. Serum plasma samples are obtained at regular intervals and assayed for parent drug (or occasionally metabolite) concentrations. For a pharmacokinetic comparison, the plasma concentration data are used to assess key pharmacokinetic parameters such as area under the plasma concentration-time curve (AUC), peak concentration (Cₘₐₓ) and time to peak plasma concentration (Tₘₐₓ).

Certain specific aspects and embodiments of the invention will be further described in the following examples, which are provided solely for purposes of illustration and are not intended to limit the scope of the invention in any manner.

### EXAMPLE 1

### Pioglitazone Hydrochloride 15 mg Tablets

| Component | mg/Tablet | |
|---|---|---|
| Pioglitazone hydrochloride* | 16.54 | |
| Lactose monohydrate | 55.03 | |
| Low substituted hydroxypropyl | 2.4 | |
| cellulose (HPC LH 21)# | | |
| Hydroxypropyl cellulose (Klucel | 2.4 | |
| LF)** | | |
| Croscarmellose sodium | 2.83 | |
| Water | 0.02 | mL |
| Magnesium stearate | 0.8 | |

*D₁₀ 2 μm, D₅₀ 12 μm, D₉₀ 32 μm, D_{[4,3]} 15 μm.
#HPC LH 21 is supplied by Shin-Etsu Chemical (Japan).
**Klucel LF is manufactured by Hercules Inc.

### Manufacturing Process:

1) Pioglitazone hydrochloride, lactose monohydrate, HPC LH 21 and croscarmellose sodium were sifted through a 40 mesh sieve and Klucel LF through a 20 mesh sieve.
2) Sifted materials of step 1 were loaded into a rapid mixer granulator and mixed for 15 minutes at fast impeller speed and chopper off.
3) The dry mix of step 2 was granulated using water for about 2 minutes with impeller fast speed and chopper off. Mixed at impeller fast speed and chopper slow speed for a period of 6 minutes until a suitable granular mass was obtained. The wet granules were unloaded into the bowl of a fluid bed drier.
4) The wet granules were dried at an inlet air temperature of 65±5° C. until the loss on drying obtained was within the range of 1.0%-2.5% by weight, determined using an infrared/halogen moisture analyzer.
5) The dried granules of step 4 were sifted through a 20 mesh sieve.
6) Retains of 20 mesh sieve were milled in a comminuting mill fitted with a 1.5 mm screen at a medium speed and knives forward.
7) The milled granules of step 6 were sifted through a 20 mesh sieve.
8) The sifted materials of step 5 and step 7 and magnesium stearate (sifted through a 60 mesh sieve) were loaded into a double cone blender and blended for 5 minutes.
9) The blend was compressed into tablets.
10) Tablets were packaged into high-density polyethylene (HDPE) containers and in aluminium foil pouch packs.

In vitro dissolution analysis of samples prepared in Example 1 was performed in USP apparatus II (paddles) at 75 rpm and compared with that of a reference commercial product (ACTOS® tablets 15 mg). The results are given in Table 1.

Medium: pH 2.0 buffer (degassed) [pH 2 buffer is prepared by mixing 250 mL of 0.3 M KCl and 50 mL of 0.2 M HCl in 1000 mL of purified water)

Volume: 900 mL.

Temperature: 37±0.5° C.

TABLE 1

Cumulative %
Drug Dissolved

| Time | | ACTOS ® |
|---|---|---|
| (minutes) | Example 1 | Tablets 15 mg |
| 5 | 75 | 90 |
| 10 | 94 | 95 |
| 15 | 100 | 99 |
| 30 | 102 | 102 |

### EXAMPLE 2

### Pioglitazone Hydrochloride 30 mg Tablets

| Component | mg/Tablet | |
|---|---|---|
| Pioglitazone hydrochloride* | 33.08 | |
| Lactose monohydrate | 110.6 | |
| Low substituted hydroxypropyl | 4.8 | |
| cellulose (HPC LH 21) | | |
| Hydroxypropyl cellulose (Klucel | 4.8 | |
| LF) | | |
| Croscarmellose sodium | 5.66 | |
| Water | 0.03 | mL |
| Magnesium stearate | 1.6 | |

*D₁₀ 2 μm, D₅₀ 12 μm, D₉₀ 32 μm, D_{[4,3]} 15 μm.

Manufacturing process: The composition was prepared in the same manner as described in Example 1.

In vitro dissolution analysis of samples prepared in Example 2 was performed in USP apparatus 11 (paddles) at 75 rpm and compared with that of a commercial reference product (ACTOS® tablets 30 mg). The results are given in Table 2.

Medium: pH 2.0 buffer with 0.3 M KCl (degassed).

Volume: 900 mL.

Temperature: 37±0.5° C.

TABLE 2

Cumulative % Drug Dissolved

| Time | | ACTOS ® Tablets |
|---|---|---|
| (minutes) | Example 2 | 30 mg |
| 5 | 81 | 83 |
| 10 | 91 | 92 |
| 15 | 95 | 95 |
| 30 | 98 | 98 |

### EXAMPLE 3

### Pioglitazone Hydrochloride 45 mg Tablets

| Component | mg/Tablet | |
|---|---|---|
| Pioglitazone hydrochloride* | 49.61 | |
| Lactose monohydrate | 168.99 | |
| Calcium carboxy methyl cellulose | 16 | |
| Hydroxypropyl cellulose (Klucel LF) | 3 | |
| Water | 0.05 | mL |
| Magnesium stearate | 2.4 | |

*D₁₀ 1.216 μm, D₅₀ 6.695 μm, D₉₀ 17.364 μm, D_{[4,3]} 8.191 μm.

Manufacturing process:
1) Pioglitazone hydrochloride, lactose monohydrate, and calcium carboxy methylcellulose were sifted through a 40 mesh sieve and Klucel LF through a 20 mesh sieve.
2) The sifted materials of step 1 were loaded into a rapid mixer granulator and mixed for about 15 minutes at fast impeller speed and chopper off.
3) The dry mix of step 2 was granulated using water for about 2 minutes with impeller fast speed and chopper off. Mixed at impeller fast speed and chopper slow speed for a period of about 6 minutes until a suitable granular mass was obtained. Granules were unloaded into the bowl of a fluid bed drier.
4) The wet granules of step 3 were dried at an inlet air temperature of 65±5° C. until the loss on drying of granules obtained was within the range of 1%-2.5% by weight, as determined using an infrared/halogen moisture analyzer
5) The dried granules of step 4 were sifted through a 20 mesh sieve.
6) The retained material on the 20 mesh sieve was milled in a comminuting mill fitted with a 1.5 mm screen at a medium speed and knives forward.
7) The milled granules of step 6 were sifted through a 20 mesh sieve.
8) The sifted materials of step 5 and step 7 and magnesium stearate (sifted through a 60 mesh sieve) were placed into a double cone blender and blended for 5 minutes.
9) The blend was compressed into tablets.
10) The tablets were packed in HDPE containers and in aluminium foil pouch packs.

In vitro dissolution analysis of samples prepared in Example 3 was performed in USP apparatus 11 (paddles) at 75 rpm and compared with that of a commercial reference product, ACTOS tablets 45 mg. The results are given in Table 3.

Medium: pH 2.0 buffer (degassed).

Volume: 900 mL.

Temperature: 37±0.5° C.

TABLE 3

Cumulative % Drug Dissolved

| Time | | ACTOS ® Tablets |
|---|---|---|
| (minutes) | Example 3 | 45 mg |
| 5 | 83 | 85 |
| 10 | 92 | 94 |
| 15 | 98 | 98 |
| 30 | 102 | 102 |

The pharmacokinetic parameters Cₘₐₓ(maximum concentration of drug in the plasma), AUC₀₋ₜ (area under the curve from time 0 to time t (96 hours)), and AUC_{0-∝} (area under the curve from time 0 to time infinity) have been determined for the above preparation and the mean results are given in Table 4.

T represents test product, i.e., Example 3.

R represents the reference product, i.e., ACTOS tablets 45 mg.

Study design: Open label, randomized, cross-over single dose study under fasting conditions.

Number of subjects: 37.

TABLE 4

| Cₘₐₓ (T/R) % | AUC₀₋ₜ (T/R) % | AUC_{0-∝} (T/R) % |
|---|---|---|
| 85.70 | 89.25 | 89.5 |

### EXAMPLE 4

### Pioglitazone Hydrochloride 45 mg Tablets with Different Particle Size Distributions of Pioglitazone Hydrochloride

Trial 1 (comparative): Particle size distribution of pioglitazone hydrochloride used was D₁₀ 1.643 μm, D₅₀ 13.336 μm, Dgo 49.42 μm, D_{[4,3]} 20.876 μm.

Trial 2: Particle size distribution of pioglitazone hydrochloride used was D₁₀ 2 μm, D₅₀ 12 μm, D₉₀ 32 μm, D_{[4,3]} 15 μm.

| Component | mg/Tablet | |
|---|---|---|
| Pioglitazone hydrochloride | 49.61 | |
| Lactose monohydrate | 165.09 | |
| Low substituted hydroxypropylcellulose | 7.2 | |
| (HPC LH 21) | | |
| Hydroxypropyl cellulose (Klucel LF) | 7.2 | |
| Croscarmellose sodium | 8.5 | |
| Water | 0.06 | mL |
| Magnesium stearate | 2.4 | |

Manufacturing process: The compositions were prepared in the same manner as described in Example 1.

Pharmacokinetic parameters of pioglitazone hydrochloride 45 mg tablets of Trial 1 and Trial 2 were compared. The pharmacokinetic parameters determined were Cₘₐₓ, AUC_{0-T} and AUC_{0-∝}. The data are given in Table 5.

T represents test product.

R represents the reference product, i.e., ACTOS tablets 45 mg.

Study design: Single dose crossover study design, fasting conditions.

Number of subjects: 37.

TABLE 5

| Tablets | Cₘₐₓ (T/R) % | AUC₀₋ₜ (T/R) % | AUC_{0-∝} (T/R) % |
|---|---|---|---|
| Trial 1 | 89.05 | 96.70 | 95.4 |
| Trail 2 | 102.7 | 104.2 | 103.1 |

Tablets from trial 2 and a commercial reference product were subjected to dissolution testing using the following conditions:

Medium: pH 2 buffer with 0.3M KCl.

Agitation: 75 rpm.

Apparatus USP Apparatus Type II.

Volume: 900 mL.

Reference product: ACTOS® 45 mg tablets.

Cumulative % Drug Dissolved

| Time | | ACTOS ® Tablets |
|---|---|---|
| (minutes) | Trial 2 | 45 mg |
| 10 | 79 | 89 |
| 20 | 88 | 94 |
| 30 | 93 | 97 |
| 45 | 95 | 99.5 |
| 60 | 97 | 99.3 |

### EXAMPLE 5

### Comparative Bioavailability Data for Pioglitazone Hydrochloride Tablets Containing Pioglitazone of Defined Particle Size

Pharmacokinetic parameters are tabulated in Table 6.

T represents test product, i.e., Trial 2 of Example 4.

R represents reference product, i.e., ACTOS tablets 45 mg.

Study design: Open label randomized cross-over single dose study under fasting conditions.

Number of subjects: 55.

TABLE 6

| | Example | ACTOS | T/R | 90% Confidence |
|---|---|---|---|---|
| | 4, Trial 2 | Tablets 45 mg | Ratio | Interval of T/R |
| Parameter | (T) | (R) | (%) | Ratio |
| Cₘₐₓ (ng/mL) | 1284.890 | 1250.580 | 102.7 | 93.19-113.28 |
| AUC₀₋ₜ | 14820.774 | 14229.735 | 104.2 | 95.60-113.47 |
| (ng · hour/mL) | | | | |
| AUC_{0-∝} | 15317.724 | 14862.097 | 103.1 | 94.97-111.86 |
| (ng · hour/mL) | | | | |

Based on these results, therapeutically equivalent ranges of plasma pioglitazone from administration of a single dose to humans were calculated to give the following:

Cₘₐₓ about 1028 ng/mL to about 1606 ng/mL.

AUC_{0-∞}T about 11857 ng·hour/mL to about 18526 ng·hour/mL.

AUC_{0-∞} about 12254 ng·hour/mL to about 19147 ng·hour/mL.

### EXAMPLE 6

### Pioglitazone Hydrochloride 45 mg Capsules

| Component | mg/Capsule |
|---|---|
| Pioglitazone hydrochloride | 49.61 |
| Lactose monohydrate | 187.49 |
| Disodium edetate | 0.5 |
| Magnesium stearate | 2.4 |

Manufacturing procedure:
1) Sift pioglitazone hydrochloride, disodium edetate, and lactose monohydrate through a 40 mesh sieve.
2) Sift magnesium stearate through a 60 mesh sieve.
3) Mix step 1 and step 2.
4) Load step 3 into a double cone blender and blend for 5 minutes.
5) Fill the final blend into empty hard gelatin capsule shells.

1. Pioglitazone or a salt thereof having a particle size distribution wherein D₉₀ is about 25 μm to about 40 μm.

2. Pioglitazone or a salt thereof of claim 1, wherein D_{[4,3]} is about 12 μm to about 17 μm.

3. Pioglitazone or a salt thereof of claim 1, wherein D₅₀ is about 8 μm to about 16 μm.

4. Pioglitazone or a salt thereof of claim 1, wherein D₁₀ is about 1 μm to about 4 μm.

5. Pioglitazone or a salt thereof of claim 1, comprising pioglitazone hydrochloride.

6. A pharmaceutical formulation comprising pioglitazone or a salt thereof of claim 1, and at least one pharmaceutical excipient.

7. Pioglitazone or a salt thereof having a particle size distribution wherein D₉₀ is about 25 μm to about 40 μm, D₅₀ is about 8 μm to about 16 μm, D₁₀ is about 1 μm to about 4 μm, and D_{[4,3]} is about 12 μm to about 17 μm.

8. Pioglitazone or a salt thereof of claim 7, comprising pioglitazone hydrochloride.

9. A pharmaceutical formulation comprising pioglitazone or a salt thereof of claim 7, and at least one pharmaceutical excipient.

10. The pharmaceutical formulation of claim 9, wherein at least about 70% of contained pioglitazone dissolves within about 30 minutes upon immersion in a buffer having a pH about 2.

11. The pharmaceutical formulation of claim 9, producing plasma Cₘₐₓ values about 1,000 ng/mL to about 1,700 ng/mL after administration of a 45 mg pioglitazone dose to healthy humans.

12. The pharmaceutical formulation of claim 9, producing plasma AUC_{0-∞} values about 10,000 ng·hour/mL to about 19,000 ng·hour/mL after administration of a single 45 mg pioglitazone dose to healthy humans.

13. The pharmaceutical formulation of claim 9, producing plasma AUC_{0-∝} values about 12,000 ng·hour/mL to about 20,000 ng·hour/mL after administration of a single 45 mg pioglitazone dose to healthy humans.

14. A pharmaceutical formulation comprising pioglitazone hydrochloride having a particle size distribution wherein D₉₀ is about 25 μm to about 40 μm, D₅₀ is about 8 μm to about 16 μm, D₁₀ is about 1 μm to about 4 μm, and D_{[4,3]} is about 12 μm to about 17 μm, and at least one pharmaceutical excipient, wherein at least about 70% of contained pioglitazone dissolves within about 30 minutes upon immersion in a buffer having a pH about 2.

15. The pharmaceutical formulation of claim 14, producing plasma Cₘₐₓ values about 1,000 ng/mL to about 1,700 ng/mL after administration of a 45 mg pioglitazone dose to healthy humans.

16. The pharmaceutical formulation of claim 14, producing plasma AUC_{0-∝} values about 10,000 ng·hour/mL to about 19,000 ng·hour/mL after administration of a single 45 mg pioglitazone dose to healthy humans.

17. The pharmaceutical formulation of claim 14, producing plasma AUC_{0-∝} values about 12,000 ng·hour/mL to about 20,000 ng·hour/mL after administration of a single 45 mg pioglitazone dose to healthy humans.
